(19) United States
(12) Patent Application Publication
(10) Pub. No.: US 2026/0184677 A1
(43) Pub. Date: Jul. 2, 2026

(54) METHOD FOR PREPARING A COMPOSITION IN POWDER FORM COMPRISING AT LEAST ONE SOLID ORGANIC PEROXIDE

(71) Applicant: ARKEMA FRANCE, Colombes (FR)

(72) Inventors: Juergen LOHR, Günzburg (DE); Albert BLUM, Günzburg (DE); Alfredo DEFRANCISCI, Pierre-Benite (FR); Mathieu BAILLET, Pierre-Benite (FR)

(73) Assignee: ARKEMA FRANCE, Colombes (FR)

(21) Appl. No.: 18/867,698

(22) PCT Filed: Jul. 28, 2023
(86) PCT No.: PCT/EP2023/071026

§ 371 Date: Nov. 20, 2024

(30) Foreign Application Priority Data
Aug. 1, 2022 (FR) FR2207963

(51) Int. Cl. C07C 407/00; C08K 3/013; C08K 5/14

(52) U.S. Cl. CPC C07C 407/006; C08K 3/013; C08K 5/14

(57) ABSTRACT

A method for producing a powdered composition including at least one solid organic peroxide displaying the assets of having a reduced water-content and being easily flowable. Also, a powdered composition which is obtainable from the method. Also, the use of said powdered composition as polymerization initiator for acrylic resins or unsaturated polyester resins, preferably acrylic resins, or as a polymer modifier, for example as a polymer crosslinking agent, grafting agent or rheology modifier, preferably as a polymerization initiator for acrylic resins or unsaturated polyester resins. Also, the use of said powdered composition for road marking, chemical anchoring, water proofing and floor coating applications, preferably road marking.

### FIELD OF THE INVENTION

The present invention deals with a method for producing a powdered composition comprising at least one solid organic peroxide advantageously displaying the assets of having a reduced water-content and being easily flowable.

The invention also pertains to a powdered composition which is obtainable from the method as defined hereafter.

The instant invention further relates to the use of said powdered composition as polymerization initiator for acrylic resins or unsaturated polyester resins, preferably acrylic resins, or as a polymer modifier, for example as a polymer crosslinking agent, grafting agent or rheology modifier, preferably as a polymerization initiator for acrylic resins or unsaturated polyester resins.

The invention still further relates to the use of said powdered composition for road marking, chemical anchoring, water proofing and floor coating applications, preferably road marking.

### BACKGROUND OF THE INVENTION

Acrylic resins and polyester resins are currently used in many different technical fields for industrial or decorative applications, for example for the preparation of paints, namely for road marking, the protection of structures and the waterproofing of floors, the manufacture of chemical seals (also known as chemical anchoring) in order to fasten objects of varying sizes on numerous surfaces, the production of composite materials in association with reinforced fibers, the manufacture of marbles, natural or synthetic, as well as sealants especially used for car bodyworks repair.

Acrylic resins are mainly valued by users for their fast setting, even at low temperatures, for instance at temperatures around 10° C., their strong adhesion to myriad types of substrates as well as their high mechanical resistance, whereas polyester resins exhibit very good chemical resistance, especially in acid environments, good properties in terms of mechanical resistance and swift commissioning.

Such resins also display the advantage that they can be applied manually or by spraying, namely by implementing various spraying techniques, to several types of surfaces.

Acrylic resins and polyester resins are mostly obtained by polymerization, in particular by free-radical polymerization of one or more acrylic monomers, identical or different, in the presence of organic peroxides, in liquid or solid form, playing the role of polymerization initiators.

A thorny aspect in the use of organic peroxides lies in the fact that they are usually highly unstable species when heated as they decompose relatively easily under the action of a small amount of heat. Thus, in case of uncontrolled rise of their storage temperature, some organic peroxides can undergo a self-accelerated exothermic decomposition leading to fires and/or violent explosions. In addition, under these conditions, some of these organic peroxides release combustible vapors that can react with any source of ignition, which can significantly increase or even accelerate the risk of bursting explosion.

Indeed, such organic peroxides have a Self-Accelerating Decomposition Temperature (SADT) which corresponds to the lowest temperature at which an uncontrolled reaction occurs, that is to say a self-accelerating decomposition in its packaging. In other words, the self-accelerating decomposition temperature SADT represents the temperature at which the chemical process leading to uncontrolled decomposition, possibly accompanied by self-combustion and explosion phenomena, begins.

Such a risky behavior is therefore incompatible with the rules in force for the transport and storage of dangerous goods. It is particularly important to take adequate stringent precautionary measures when storing and handling such organic peroxides, especially by ensuring that they remain at temperatures below their self-accelerating decomposition temperature SADT in order to minimize the risks of fire and/or uncontrolled decomposition. Hence, in some cases, such precautionary measures imply maintaining the organic peroxides at a storage temperature that is preferably at least 10° C., and even more preferably at least 20° C., below the self-accelerating decomposition temperature SADT.

In order to overcome these various disadvantages, such organic peroxides can be diluted or mixed with inert products, for example non-reactive products in contact with said organic peroxides, called phlegmatizing agents. Especially, phlegmatizing agents make it possible to limit the effects of uncontrolled decomposition of organic peroxides and consequently to reduce the various risks associated with their handling.

As of result, organic peroxides may be solubilized in a liquid phlegmatizer or mixed with a solid inert phlegmatizer, or even being implemented as an emulsion in the liquid phlegmatizer. By way of example, solid organic peroxides can be blended with water in order to form a water-wet powder.

Although the presence of water in such solid organic peroxide formulations can mitigate safety issues, it may also disturb or even hinder their final application. For instance, the presence of water can cause the formation of several small air bubbles or lumps at the surface of the acrylic paint during polymerization which may hamper its use. In addition, the presence of water in the solid organic peroxide formulation hinders its flow properties, making it more susceptible to caking during transport and storage.

Moreover, the implementation of a drying process to evaporate the aqueous phase contained in solid organic peroxide formulations can be fastidious as it requires to heat such formulations at a temperature close to or higher than the self-accelerating decomposition temperature SADT of the organic peroxide causing its chemical degradation. In other words, there is a fair risk that the implementation of such drying process might trigger the safety issues previously described.

Accordingly, it remains a real need to provide a method for producing a powdered composition comprising a solid organic peroxide displaying a low water-content and being easily flowable, without triggering during its implementation the safety issues linked to the chemical uncontrolled decomposition of the organic peroxide. In other words, one of the purposes of the present invention is to safely produce a flowable product comprising a solid organic peroxide which is ready for use and can be implemented without hindrance in its intended final application.

### DESCRIPTION OF THE INVENTION

The present invention namely results from the unexpected findings, by the inventors, that the implementation of a process as described hereafter is able to achieve the aforementioned goals.

Therefore, the present invention relates to a method for producing a composition in powder form, comprising the steps of:
a) mingling at least one organic solvent, at least one solid organic peroxide and an aqueous phase,
b) reducing the aqueous phase from said mixture,
c) mixing at least one inert filler, preferably at least two inert fillers, with the ensuing composition in order to obtain a powdered composition comprising said solid organic peroxide.

The method according to the present invention makes it possible to safely obtain a flowable powdered composition comprising at least one solid organic peroxide which is ready to be used and can be implemented without hindrance in its final application.

In other words, the method of the present invention leads to a powdered composition, preferably having fine particles, that has the assets of being easily flowable and exhibiting a low water-content.

The implementation of such method allows to rein in the risks linked to the chemical uncontrolled decomposition of the organic peroxide, especially by not carrying out a drying step for removing the aqueous phase in the composition comprising said organic peroxide.

It bespeaks that the method of the invention minimizes the risks of exothermic decomposition during the preparation of the powdered composition.

The method according to the present invention has the further advantage of producing a powdered composition comprising at least one solid organic peroxide that is ready to be mixed with acrylic or polyesters resins as it does not require to further eliminate its residual water content. As a matter of fact, the residual water content of the obtained composition does not hamper its final application.

Moreover, the method can be easily optimized and monitored on a lab-scale as well as on a large-scale production. Indeed, the process according to the invention is easy to implement and consumes a low amount of energy.

In other words, the method according to the present invention has the advantage of avoiding a drying step, which is energy-intensive and a safety risk relevant.

The present invention also deals with a composition in a powder form comprising:
at least one solid organic peroxide,
at least one inert filler, preferably at least two inert fillers,
at least one organic solvent, and
an aqueous phase, preferably water.

The composition according to the present invention is a non-caking powder having excellent free-flowing properties.

Indeed, the powdered composition according to the present invention has the advantage of having a poured cone height preferably equal to a lower than 3 cm. The lower the poured cone height, the better the flowability of the powder.

Measurement of the poured cone height under defined conditions of the powdered composition according to the present invention can be carried out by a Pfrengle test according to DIN 53 916.

The powdered composition according to the present invention also displays the advantage of generating little or no dust.

The composition as defined exhibits the desired average particle size and a homogeneous particle size distribution while remaining stable, both in storage and during handling.

The composition according to the present invention advantageously fulfils the application issues, especially on road marking.

The instant invention further relates to the use of said powdered composition as polymerization initiator, in particular for acrylic resins or unsaturated polyester resins, preferably acrylic resins, or as a polymer modifier, for example as a polymer crosslinking agent, grafting agent or rheology modifier, preferably as polymerization initiator for acrylic resins or unsaturated polyester resins, especially acrylic resins.

Indeed, the powdered composition according to the invention proves to be compatible with the acrylic resins and polyester resins obtained, preferably with the acrylic monomers, which minimizes for example the risks of occurrence of air bubbles when obtaining such resins, in particular when obtaining an acrylic paint.

Another subject-matter of the invention aims at the use of said composition for road marking, chemical anchoring, water proofing and floor coating applications, preferably road marking.

Other subjects and characteristics, aspects and advantages of the invention will emerge even more clearly on reading the description and the examples that follow.

In the text herein below, and unless otherwise indicated, the limits of a range of values are included in that range, in particular in the expressions “between” and “ranging from . . . to . . . ”.

Moreover, the expression “at least one” used in the present description is equivalent to the expression “one or more”.

In the text herein below, the terms “peroxyketal” and “perketal” are equivalent.

Furthermore, the term “polymer” encompasses “homopolymers” and “copolymers”, where the term “copolymers” refers to a polymer comprised of at least two different monomers in polymerized form. For example, a copolymer in accordance with the present disclosure may be a polymer comprising two different monomers, a terpolymer is a polymer comprising three different monomers or more.

### Composition

As previously detailed, the present invention deals with a composition in powder form comprising at least one solid organic peroxide, at least one organic solvent, at least one inert filler, preferably at least two inert fillers, and an aqueous phase, preferably water.

According to the present invention, the feature “solid organic peroxide” is to be understood that said organic peroxide is in a solid state at room temperature and under atmospheric pressure (about 1 bar or 1.013×10⁵ Pa).

For the purposes of the present invention, the terms “room temperature” means a temperature ranging from 15° C. to 27° C., preferably from 20° C. to 25° C.

Preferably, the solid organic peroxide is selected from the group consisting of solid diacyl peroxides, solid peroxydicarbonates, solid ketone peroxides, solid peroxy esters, solid hydroperoxides, solid dialkyl peroxides and mixtures thereof, preferably selected from the group consisting of solid diacyl peroxides, solid peroxydicarbonates and mixtures thereof, more preferably selected from the group consisting of solid diacyl peroxides.

The solid diacyl peroxides are preferably selected from the group consisting of dibenzoyl peroxide, di (2-methylbenzoyl) peroxide, di(methoxybenzoyl) peroxide, di (2-methoxycarbonylbenzoyl) peroxide, di (2-benzylbenzoyl) peroxide, di (4-fluorobenzoyl) peroxide, di (3-chlorobenzoyl) peroxide, di (4-chlorobenzoyl) peroxide, di (2,4-dichlorobenzoyl) peroxide, dibenzoyl diperoxy adipate, benzoyl octadecanoyl peroxide, dilauroyl peroxide, dihexadecanoyl peroxide, di (chloroacetyl) peroxide, and di (3-carboxypropionyl) peroxide.

The solid peroxydicarbonates are preferably dialkyl peroxydicarbonates, particularly selected from the group consisting of dibenzyl peroxydicarbonate, dicyclohexyl peroxydicarbonate, di (cis-3, 3,5-trimethylcyclohexyl) peroxydicarbonate, di (4-t-butylcyclohexyl) peroxydicarbonate, dibornyl peroxydicarbonate, di (2-phenoxyethyl) peroxydicarbonate, di-n-tridecyl peroxydicarbonate and di-n-hexadecyl peroxydicarbonate.

The solid ketone peroxides are preferably selected from the group consisting of di (1-hydroxycyclohexyl) peroxide, 1-hydroxycyclohexyl-1-hydroperoxycyclohexyl, di (hydroperoxycyclohexyl) peroxide and 3,5-dihydroxy-3,5-dimethyl-1,2-dioxolane.

The solid peroxyesters are preferably selected from the group consisting of di-t-butyl diperoxyterephthalate, di-t-butyl diperoxysuccinate, di-t-butyl diperoxyadipate, di-t-butyl diperoxyphthalate, t-butyl peroxy-(3-carboxypropionate), t-butylperoxy-(3-carboxy-2-propenoate) and 2,5-dimethyl-2,5-di (benzoylperoxy) hexane.

The solid alkyl hydroperoxides are preferably selected from the group consisting of 2,5-dimethyl-2,5-dihydroxyperoxyhexane, 2,5-dimethyl-2,5-dihydroxyl)oxy-3-hexane, 2,7-dimethyl-2,7-dihydroperoxy-3,5-octadiyne, 1,4-di(1-methyl-1-hydroxyperoxyethyl)benzene, and 1,3,5,-tri (1-methyl-1-hydroperoxyethyl)benzene.

The solid dialkyl peroxides are preferably selected from the group consisting of di-cumyl peroxide, 1,4-di [1-methyl-1-(t-butylperoxy) ethyl]benzene, 1,3-di-[1-methyl-1-(t-butylperoxy)-ethyl]-benzene and di (isopropyl-cumyl) peroxide.

Preferably, the solid organic peroxide is selected from the group consisting of solid diacyl peroxides, solid peroxydicarbonates and mixtures thereof. Even more preferably, the solid organic peroxide is selected from the group consisting of solid diacyl peroxides, especially dibenzoyl peroxide.

Preferably, the solid organic peroxide is dibenzoyl peroxide.

The mixture preferably includes dibenzoyl peroxide such as the product sold under the trade name Luperox® A75 by the company Arkema.

The solid organic peroxide is preferably present in a content ranging from 10 to 60% by weight, relative to the total weight of the powdered composition.

According to a preferred embodiment, the solid organic peroxide is present in a content ranging from 20 to 60% by weight, preferably from 30 to 55% by weight, more preferably from 40 to 55% by weight, relative to the total weight of the powdered composition.

Preferably, the organic solvent has a solubility lower than 5 mg/l, preferably lower than 1 mg/l, preferably lower than 0.1 mg/l in water at room temperature, particularly at a temperature ranging from 15° C. to 27° C., preferably from 20° C. to 25° C., under atmospheric pressure (about 1 bar or 1.013×10⁵ Pa).

Preferably, the solid organic peroxide has a solubility lower than 5 wt %, preferably lower than 1 wt %, preferably lower than 0.1 wt % in the organic solvant at room temperature, particularly at a temperature ranging from 15° C. to 27° C., preferably from 20° C. to 25° C., under atmospheric pressure (about 1 bar or 1.013×10⁵ Pa).

Preferably, the organic solvent has a solubility lower than 5 mg/l, preferably lower than 1 mg/l, preferably lower than 0.1 mg/l in water at room temperature particularly at a temperature ranging from 15° C. to 27° C., preferably from 20° C. to 25° C., under atmospheric pressure (about 1 bar or 1.013×10⁵ Pa), and the solid organic peroxide has a solubility lower than 5 wt %, preferably lower than 1 wt %, preferably lower than 0.1 wt % in the organic solvant at room temperature, particularly at a temperature ranging from 15° C. to 27° C., preferably from 20° C. to 25° C., under atmospheric pressure (about 1 bar or 1.013×10⁵ Pa).

The organic solvent is namely liquid up to a temperature strictly lower than the self-accelerating decomposition temperature SADT of said organic peroxide.

The term “self-accelerating decomposition temperature SADT of organic peroxide” in the sense of the present invention means the lowest temperature at which an exothermic decomposition of the organic peroxide starts in its packaging, preferably according to the UN H.4 test, as described in the UN's Manual of Tests and Criteria, 7th revised edition of 2019.

For the purposes of the present invention, the feature “the organic solvent being liquid up to a temperature strictly lower than the self-accelerating decomposition temperature SADT of said organic peroxide” is to be understood that the organic solvent remains in a liquid state up to a temperature strictly lower than the lowest temperature at which an exothermic decomposition of the organic peroxide starts in its packaging, preferably according to the UN H.4 test.

According to a preferred embodiment, the organic solvent (or the mixture of organic solvents) is in a liquid state at a temperature between 4° C. up to a temperature strictly lower than the self-accelerating decomposition temperature SADT of said organic peroxide, preferably at a temperature between 15° C. up to a temperature strictly lower than the self-accelerating decomposition temperature SADT of said organic peroxide.

According to a preferred embodiment, the organic solvent is in a liquid state at a temperature between 15° C. to at least 10° C. lower, preferably at least 20° C. lower, than the self-accelerating decomposition temperature SADT of said solid organic peroxide.

According to a preferred embodiment, the organic solvent is in a liquid state at a temperature ranging from 15° C. up to a temperature strictly lower than 75° C., preferably from 25° C. to 65° C., more preferably from 35° C. to 60° C., even more preferably from 45° C. to 55° C.

Advantageously, the organic solvent is a non-phthalate organic solvent, preferably non-phthalate plasticizer.

Advantageously, the organic solvent exhibits a very good toxicological profile and has been approved for sensitive applications by the competent authorities.

Especially, the organic solvent has the benefit of being approved for food contact applications.

Preferably, the organic solvent is food approved.

Preferably, when the organic solvent has a solubility lower than 5 mg/l in water at room temperature, preferably lower than 1 mg/l in water at room temperature, preferably lower than 0.1 mg/l in water at room temperature and the solid organic peroxide has a solubility lower than 5 wt % in the organic solvant at room temperature, preferably lower than 1 wt % in the organic solvent at room temperature, preferably lower than 0.1 wt % in the organic solvant at room temperature, the organic solvent can be selected in the group consisting of esters, in particular esters of mono, di, tri, poly carboxylic acids reacted with mono, di, tri, poly hydric alcohols, alcohols, alkanes and mineral oils.

The organic solvent is preferably selected from the group consisting of alkyl diacid esters, cyclohexanedicarboxylates, esters based on glycols, polyglycols and polyhydric alcohols, and mixtures thereof, preferably from the group consisting of cyclohexanedicarboxylates and alkyl diacid esters and mixtures thereof.

Suitable alkyl diacid esters can be selected from C₄-C₁₂ alkyl dicarboxylic acids such as adipic, sebacic, azelaic, and maleic acids.

The alkyl diacid esters can be selected from C₄-C₁₂ alkyl dicarboxylic acids preferably selected from the group consisting of Bis (2-ethylhexyl) adipate (DEHA), Dimethyl adipate (DMAD), Monomethyl adipate (MMAD), Dioctyl adipate (DOA), Dibutyl sebacate (DBS), Dibutyl maleate (DBM), Diisobutyl maleate (DIBM), Dioctyl sebacate (DOS), and mixtures thereof.

Suitable esters based on glycols, polyglycols and polyhydric alcohols can be selected from the group consisting of poly(ethylene glycol) mono- and di-esters, cyclohexanedimethanol esters, sorbitol derivatives; and triethylene glycol dihexanoate, diethylene glycol di-2-ethylhexanoate, tetraethylene glycol diheptanoate, and ethylene glycol dioleate, and mixtures thereof.

Preferably, the organic solvent is selected among the group consisting of cyclohexanedicarboxylates, alkyl diacid esters and mixtures thereof, especially from the group consisting of cyclohexanedicarboxylates, C₄-C₁₂ alkyl dicarboxylic acids, and mixtures thereof.

Preferably, the organic solvent is selected from cyclohexanedicarboxylates, more preferably diisononyl-1,2-cyclohexanedicarboxylate, for example diisononyl-1,2-cyclohexanedicarboxylate available under the commercial name HEXAMOLL DINCH® from BASF.

HEXAMOLL DINCH has the advantage that it can be used for sensitive human applications, such as food packaging, medical products and toys.

Especially, HEXAMOLL DINCH has the advantage that it can be used for food contact applications.

The organic solvent may be present in the powdered composition at a concentration ranging from 5 to 50% by weight, preferably from 10% to 30% by weight, even more preferably from 15% to 25% by weight, relative to the total weight of said powdered composition.

As previously detailed, the composition according to the present invention further comprises at least one inert filler, preferably at least two inert fillers.

The inert filler is preferably selected from the inert fillers that are soluble in acrylic and/or polyester resins, preferably soluble in acrylic resins, at room temperature, particularly at a temperature ranging from 15° C. to 27° C., preferably from 20° C. to 25° C.

According to the present invention, the term “inert filler” is to be understood that the filler does not trigger the exothermic decomposition of said organic peroxide. In other words, the presence of the filler does not accelerate the exothermic decomposition of said organic peroxide.

Preferably, the inert filler is different from carbon black.

Preferably, the inert filler is chosen among the group consisting of inorganic inert filler.

The inert filler is preferably selected from the group consisting of silicas, more preferably selected from the group consisting of silicas obtainable from a precipitation process.

The inert filler is preferably selected from the group consisting of hydrophobic silicas, hydrophilic silicas, and mixtures thereof.

The inert filler is preferably selected from a combination of dual-functionally silicas. Especially, the inert filler is advantageously selected from a combination of at least one hydrophobic silica and at least one hydrophilic silica.

Advantageously, the presence of a combination of at least one hydrophobic silica and at least one hydrophilic silica enhances the flowability of the powdered composition.

The inert filler is preferably selected from the silicas sold by the Evonik company under the trade names SIPERNAT®.

Especially, the inert filler may be selected from the group consisting of hydrophobic silicas, such as silicas sold under the commercial name SIPERNAT® D17, hydrophilic silicas, such as silicas sold under the commercial name SIPERNAT® 2200, and mixtures thereof.

Preferably, the inert filler is selected from a combination of at least one hydrophobic silica sold under the commercial name SIPERNAT® D17 and at least one hydrophilic silica sold under the commercial name SIPERNAT® 2200.

The composition comprises at least one inert filler, namely at least two inert fillers, preferably at a concentration ranging from 5% to 50% by weight, more preferably ranging from 10% to 30% by weight, even more preferably from 15% to 25% by weight, relative to the total weight of said powdered composition. When at least two inert fillers are present, the preferred total concentration of fillers ranges from 5% to 50% by weight, more preferably ranges from 10% to 30% by weight, even more preferably from 15% to 25% by weight, relative to the total weight of said powdered composition

The composition may further comprise at least a powder having a crystalline structure and a melt temperature ranging from 30° C. to 80° C. in order to advantageously reduce the abrasiveness of said powdered composition.

The powder having a crystalline structure and a melt temperature ranging from 30° C. to 80° C. can be selected from solid fatty acids.

The solid fatty acids are preferably linear or branched, and comprise in their structure a number of carbon atoms ranging from 6 to 20, preferably ranging from 10 to 20, more preferably ranging from 12 to 20, even more preferably from 14 to 20, especially from 16 to 20.

Preferably, the fatty acids are selected from the group consisting of lauric acid, myristic acid, capric acid, palmitic acid, stearic acid and mixtures thereof.

Even more preferably, the solid fatty acid is stearic acid, for instance stearic acid sold under the commercial name EDENOR® ST1 GS by the company EMERY.

According to a preferred embodiment, the composition comprises at least one inert filler, preferably two inert fillers, selected from silicas, and at least a powder having a crystalline structure and melt temperature ranging from 30° C. to 80° C., preferably selected from solid fatty acids, in particular stearic acid.

The aqueous phase, preferably water, may be present at a concentration of up to 10% by weight (10% by weight being included), relative to the total weight of the powdered composition, preferably at a concentration ranging from 3 to 10% by weight, preferably from 4 to 7%, relative to the total weight of the powdered composition, and even more preferably 4 to 6% by weight relative to the total weight of the powdered composition.

Preferably, the composition is a free-phthalate formulation.

According to a preferred embodiment, the composition comprises:
at least one solid organic peroxide as previously defined, preferably selected from diacyl peroxides,
at least one filler chosen from the group consisting of silicas, preferably selected from the group consisting of silicas obtainable from a precipitation process,
at least one organic solvent chosen from the group consisting of cyclohexanedicarboxylates, alkyl diacid esters and mixtures thereof, especially from the group consisting of cyclohexanedicarboxylates, C₄-C₁₂ alkyl dicarboxylic acids, and mixtures thereof, and
an aqueous phase, preferably water.

Preferably, the composition in powder form comprises:
at least one solid organic peroxide as previously defined, preferably selected from diacyl peroxides,
a combination of at least one hydrophobic silica and at least one hydrophilic silica,
at least one organic solvent chosen from the group consisting of cyclohexanedicarboxylates, C₄-C₁₂ alkyl dicarboxylic acids, and mixtures thereof,
an aqueous phase, preferably water, at a concentration up to 10% by weight, preferably ranging from 3 to 10% by weight, especially ranging from 4 to 7% by weight, and even more preferably from 4 to 6% by weight relative to the total weight of the powdered composition,
preferably at least a powder having a crystalline structure and melt temperature ranging from 30° C. to 80° C., preferably selected from the solid fatty acids, especially stearic acid.

Preferably, the composition in powder form comprises:
at least one solid organic peroxide as previously defined, preferably selected from diacyl peroxides, in a content ranging from 20 to 60% by weight, preferably from 30 to 55% by weight, more preferably from 40% to 55% by weight relative to the total weight of the powdered composition,
at least one inert filler, preferably two inert fillers, preferably selected from a combination of at least one hydrophobic silica and at least one hydrophilic silica in a content ranging from 5% to 60% by weight, preferably ranging from 10 to 40% by weight, even more preferably from 15 to 35% by weight relative to the total weight of the powdered composition,
at least one organic solvent chosen from the group consisting of cyclohexanedicarboxylates, C₄-C₁₂ alkyl dicarboxylic acids, and mixtures thereof, in a content ranging from 5 to 50% by weight, preferably ranging from 10 to 30% by weight, even more preferably from 15 to 25% by weight relative to the total weight of the powdered composition.
an aqueous phase, preferably water, at a concentration up to 10% by weight, preferably ranging from 3 to 10% by weight, especially ranging from 4 to 7% by weight, even more preferably from 4 to 6% by weight relative to the total weight of the powdered composition,
preferably at least a powder having a crystalline structure and melt temperature ranging from 30° C. to 80° C., preferably selected from the solid fatty acids, especially stearic acid.

The composition may comprise one or more of the compound chosen from the group consisting of: sodium tripolyphosphate, at least one pH modifier agent which can be chosen among alkaline agents, especially chosen from organic or mineral alkaline agents, acidifying agents, especially chosen from organic or mineral acids such as hydrochloric acid, phosphoric acid, lactic acid or citric acid, and mixtures thereof.

Preferably, the composition according to the present invention is a water-wet powder.

### Method

As previously detailed, the method according to the present invention comprises the steps of:
a) mingling at least one organic solvent as previously defined, at least one solid organic peroxide as previously defined, and an aqueous phase, preferably water,
b) reducing the aqueous phase from said mixture,
c) mixing at least one inert filler, preferably at least two inert fillers, as previously defined, with the ensuing composition in order to obtain a powdered composition comprising said solid organic peroxide.

According to step a), at least one organic solvent, at least one solid organic peroxide and an aqueous phase, preferably water, are mingled to obtain a mixture.

According to an embodiment, the organic solvent, the solid organic peroxide and the aqueous phase, preferably water, can be mingled at the same time.

According to an embodiment, the solid organic peroxide and the aqueous phase, preferably water, are first mixed and then the organic solvent is added in order to be mingled. Alternatively, the solid organic peroxide and at least part of the aqueous phase are already mixed together before step a).

According to an embodiment, the organic solvent and the aqueous phase, preferably water, are first mixed and then the solid organic peroxide is added in order to be mingled.

According to a particular embodiment, the organic solvent is added into an aqueous phase to be mixed and then the solid organic peroxide is added in order to be mingled.

According to a preferred embodiment, the aqueous phase may comprise sodium tripolyphosphate in order to prevent the mixture from sticking to device surfaces, especially surfaces of the reactor involved during the implementation of said mixture.

According to a preferred embodiment, the aqueous phase may further comprise at least one pH modifier agent which can be chosen among alkaline agents, especially chosen from organic or mineral alkaline agents, acidifying agents, especially chosen from organic or mineral acids such as phosphoric acid, lactic acid or citric acid, and mixtures thereof.

Preferably, the aqueous phase may contain at least one pH modifier agent chosen among the group consisting of mineral alkaline agents, such as sodium hydroxide (NaOH), mineral acids, and mixtures thereof.

More preferably, the aqueous phase may contain a combination of at least one alkaline agent and at least one acidifying agent, especially a combination of least one mineral alkaline agent and at least one mineral acid.

According to a preferable embodiment, a combination of least one mineral alkaline agent and at least one mineral acid can advantageously neutralize the pH of a mixture comprising at least one organic solvent chosen among the group consisting of C₄-C₁₂ alkyl dicarboxylic acids and cyclohexanedicarboxylates, especially diisononyl-1,2-cyclohexanedicarboxylate.

The pH of the mixture in step a) may be less than 9, preferably less than 8, more preferably slightly less than 8.

According to a preferred embodiment, the pH of the mixture in step a) ranges from 6 to 8, preferably from 7 to 8, even more preferably from 7.5 to less than 8.

Preferably, the aqueous phase comprises at least one pH modifier agent as previously defined and sodium tripolyphosphate.

Preferably, the organic solvent and an aqueous phase, especially containing at least one pH modifier agent and sodium tripolyphosphate, are first mixed and then the solid organic peroxide is added in order to be mingled.

According to an embodiment, step a) may be carried out up to a temperature strictly lower than the self-accelerating decomposition temperature SADT of said solid organic peroxide, preferably at least 10° C. lower, more preferably at least 20° C. lower.

According to a preferred embodiment, step a) may be carried out at a temperature ranging from 30° C. to 65° C., preferably at a temperature ranging from 40° C. to 60° C., more preferably at a temperature ranging from 45° C. to 55° C., even more preferably at a temperature ranging from 50° C. to 55° C.

According to a preferred embodiment, at least one organic solvent as previously defined and an aqueous phase as previously defined are mixed and then heated to a temperature strictly lower than the self-accelerating decomposition temperature SADT of said solid organic peroxide, particularly at a temperature ranging from 30° C. to 65° C., more particularly ranging from 40° C. to 60° C., even more particularly from 45° C. to 55° C., even more preferably at a temperature ranging from 50° C. to 55° C., before adding said solid organic peroxide.

According to this preferred embodiment, the resulting mixture can still be heated at the temperature previously defined.

As previously stated, the method further comprises a step b) of reducing the aqueous phase from said mixture, preferably in one or more steps, to obtain in particular a composition which can be in a paste form. In other words, at least part of the aqueous phase, preferably most of the aqueous phase, is separated from said mixture. Some water may remain in the mixture, preferably less than 10 wt % of water remains in said mixture after step b).

The concentration of the aqueous phase can therefore be reduced by separating said aqueous phase from said mixture in one or more steps, preferably in one step.

In particular, the aqueous phase may be separated from said mixture in one or more steps by filtration, distillation or other available methods in order to obtain a composition comprising said solid organic peroxide.

According to a preferred embodiment, the aqueous phase, preferably water, is separated from said mixture in one or more steps by filtration in order to obtain a composition comprising said solid organic peroxide.

In particular, after being mingled in step a), the mixture is cooled down, preferably to a temperature which may range from 4 to 50° C., and then the aqueous phase, preferably water, is separated from said mixture in one or more steps by filtration, preferably for a time period of at least 24 hours.

The composition resulting from the separation in step b) is therefore depleted in aqueous phase relative to the mixture provided in step a), meaning that the concentration of the aqueous phase in the resulting composition is lower than the one in the mixture implemented in step a).

Preferably, the composition resulting from the separation previously described is particularly in a paste form.

For the purposes of the present invention, the terms “pasty composition or a composition in a paste form” means that the ensued composition from the separation step has the rheological behaviour of a viscous liquid.

The pasty composition may comprise an aqueous phase, preferably water, at a concentration at a concentration ranging from 3% to 10% by weight, preferably at a concentration ranging from 4% to 7% by weight, more preferably at a concentration ranging from 5% to 7% by weight relative to the total weight of the pasty composition, The pasty composition may comprise at least said solid organic peroxide as previously defined at a concentration ranging from 20% to 75% by weight, preferably at a concentration ranging from 40% to 75% by weight, more preferably at a concentration ranging from 60% to 70% by weight relative to the total weight of the pasty composition.

The pasty composition may further comprise at least said organic solvent in a content ranging from 5 to 50% by weight, preferably in a content ranging from 10 to 40% more preferably at a concentration ranging from 15% to 30% by weight relative to the total weight of the pasty composition.

Preferably, the pasty composition comprises:
at least said organic peroxide as previously defined at a concentration ranging from 20% to 75% by weight, preferably at a concentration ranging from 40% to 75% by weight, more preferably at a concentration ranging from 60% to 70% by weight relative to the total weight of the pasty composition,
an aqueous phase, preferably water, at a concentration ranging from 3% to 10% by weight, preferably at a concentration ranging from 4% to 7% by weight, more preferably at a concentration ranging from 5% to 7% by weight relative to the total weight of the pasty composition, and
at least said organic solvent in a content ranging 5% to 50% by weight, preferably at a concentration ranging from 10% to 40% by weight, more preferably at a concentration ranging from 15% to 30% by weight relative to the total weight of the pasty composition. by weight relative to the total weight of the pasty composition.

As previously defined, the method according to the present invention further comprises a step c) of mixing at least one inert filler, preferably two inert fillers, as previously defined, with the ensuing composition in order to obtain a powdered composition comprising said solid organic peroxide.

Preferably, the inert filler is selected from a combination of at least one hydrophobic silica sold under the commercial name SIPERNAT® D17 and at least one hydrophilic silica sold under the commercial name SIPERNAT® 2200.

According to a preferred embodiment, the method may comprise adding a hydrophobic silica, such as silicas sold under the commercial name SIPERNAT® D17, in one step and adding a hydrophilic silica, such as silicas sold under the commercial name SIPERNAT® 2200, in two sequential steps into said pasty composition.

According to a preferred embodiment, the method may comprise:
i) adding into said pasty composition:
   a hydrophobic silica, such as silicas sold under the commercial name SIPERNAT® D17,
   a partial amount of hydrophilic silica, such as silicas sold under the commercial name SIPERNAT® 2200,
ii) mixing the obtained blending, especially for a time period of about 30 minutes,
iii) adding the residual amount of said hydrophilic silica into said pasty composition.

Preferably, at least one inert filler, preferably at least two inert fillers, are mixed with the composition resulting from step b), particularly the pasty composition, in a blender, especially a throwing shovel blender, usually called paddle mixers.

According to a preferred embodiment, the method can further comprise a step d) of adding of at least a powder having a crystalline structure and melt temperature ranging from 30° C. to 80° C. as defined above into the composition, preferably into the composition resulting from step b), preferably the pasty composition. Preferably, step d) is carried out before, at the same time, or after step c), and even preferably is carried out after step c).

The method can further comprise a step of limiting the particle size distribution of the obtained powdered composition comprising said organic peroxide, in particular by means of mechanical sieving on a metallic sieve.

The size and particle size distribution of the composition can be measured by dry sieving, performed by a VE 1000 sieve shaker from RETSCH, operating for 20 minutes at an oscillation angle of 1.5 mm.

The particle size of the composition advantageously is greater than 73 μm, preferably greater than 100 μm.

The particle size of the composition advantageously is lower than 1000 μm, preferably lower than 800 μm.

The method according to the present invention can lead to a powdered composition as previously defined.

Preferably, the method according to the present invention can lead to a powdered composition comprising an aqueous phase at a concentration up to 10% by weight, preferably from 3 to 10% by weight, preferably from 4 to 6% by weight, relative to the total weight of the powdered composition.

Preferably, the method according to the present invention can lead to a powdered composition comprising at least one solid organic peroxide in a content ranging from 20 to 60% by weight, preferably from 30 to 55% by weight, more preferably from 40 to 55% by weight relative to the total weight of the powdered composition.

The method according to the present invention can lead to a powdered composition comprising at least one inert filler, preferably two inert fillers, in a total content ranging from 8% to 35% by weight relative to the total weight of the powdered composition.

As previously detailed, the present invention also deals with a powdered composition which is obtainable from the method as previously defined.

### Use of the Composition

The instant invention further relates to the use of said powdered composition as polymerization initiator for acrylic resins or unsaturated polyester resins, preferably acrylic resins, or as a polymer modifier, for example as a polymer crosslinking agent, grafting agent or rheology modifier.

Preferably, the invention relates to use of said powdered composition as polymerization initiator for acrylic resins or unsaturated polyester resins, especially acrylic resins.

Another subject-matter of the invention aims at the use of said composition for road marking, chemical anchoring, water proofing and floor coating applications, preferably road marking.

### EXAMPLES

### Example 1

The purpose of the following examples (examples 1 and 2) is to study the flowing behaviour and the caking properties of a powdered commercial peroxide formulation comprising 50% by weight of dibenzoyl peroxide to which water has been added. Such commercial peroxide formulation does not contain any organic solvent.

a. Protocol

A commercial peroxide formulation in powder form sold under the name BP50 FT1 by United Initiators containing 50% by weight of dibenzoyl peroxide and 1% by weight of water has been provided.

0.15 gram of water is added to 10 grams of BP50 FT1. The components are then stirred with a spatula for 10 minutes at a temperature of 23° C. to promote mingling so that the final peroxide formulation ends up with a water content of 2.5% by weight relative to the total weight of the peroxide formulation.

The ensuing peroxide formulation is then kept in a closed beaker (closed by Parafilm) at a temperature of 23° C.

The consistency of this formulation is examined after 2 hours, 1 day, 3 days and 1 week and compared with the commercial peroxide formulation BP50 FT1 without adding water.

After each breakpoint, the peroxide formulation is stirred with a spatula to contemplate the result and then closed again and left at a temperature of 23° C.

b. Results

The findings are summarized in Table 1:

TABLE 1

| Time/after | Visual finding |
|---|---|
| T = after 2 hours | Unsatisfactory flow behaviour, lumps remaining in |
| | the peroxide formulation leading to caking |
| T = after 1 day | Flow behaviour has improved but not as good as the |
| | product BP50 FT1 without adding water. lumps are |
| | still visible. |
| T = after 3 days | Unsatisfactory caking, lumps are still visible. |
| T = after 1 week | Unsatisfactory caking, lumps are still visible. |

It bespeaks that a peroxide formulation containing 50% by weight of dibenzoyl peroxide and 2.5% by weight of water does not have satisfactory flowing behaviour and caking properties. Indeed, this formulation is a caking powdered composition being less flowable than the same formulation to which water has not been added.

### Example 2

a. Protocol

After 1 week, an additional 0.27 gram of water is added to the previously studied organic peroxide formulation (containing BP 50 FT1 and 2.5% by weight of water) to increase the total water content to 5% by weight.

After mixing for best possible homogeneity, one can observe a significant deterioration of the flowable properties of the peroxide formulation.

One can especially point out that water stays on the surface of the grains, making the powder appear wet. The grains end up being sticked to the glass and form lumps.

In the same manner as in example 1, the consistency of such formulation is again observed over a period of one week following the same breakpoints (i.e. after 2 hours, after 1 day, after 3 days and after 1 week).

The visual findings are summarized in Table 2:

TABLE 2

| Time/after | Visual finding |
|---|---|
| T = after 2 hours | Unsatisfactory flow behaviour, wetness and lumps |
| | remain which lead to caking |
| T = after 1 day | Wetness and lumps remains which lead to caking |
| T = after 3 days | Wetness and lumps remains which lead to caking |
| T = after 1 week | Wetness and lumps remains which lead to caking |

It bespeaks that such peroxide formulation containing 5% by weight of water does not exhibit a satisfactory flowing behaviour and caking properties. Indeed, this formulation is a caking powdered composition being less flowable than the same formulation to which water has not been added.

Therefore, the aforementioned examples show that powdered peroxide formulations not containing any organic solvent and having a water concentration of 2.5% or 5% by weight, relative to the total weight of the formulation, lack flowability and have caking issues.

### Examples 3

a. Protocol

In a reactor, sodium hydroxide (0.5 g) and sodium tripolyphosphate (0.5 g) are added into 1000 g of water. 170 gram of an organic solvent sold under the commercial name Hexamoll Dinch is added and the whole is then mixed to lead to a slurry.

The slurry is heated at a temperature of 55° C. before adding 636 grams of a commercial peroxide formulation sold under the name Luperox A75 (peroxide formulation comprising 75% by weight of dibenzoyl peroxide and 25% by weight of water). The ensuing mixture is then mingled at this temperature.

The mixture is then cooled down with ice and filter during 24 hours in order to separate the aqueous phase in said mixture to lead to an intermediate product having a pasty form.

The obtained intermediate product comprises 67.8% by weight of dibenzoyl peroxide.

200 grams of the obtained intermediate product are first introduced in a throwing shovel blender, generally called paddle mixers.

One or two inert fillers in the proportions given in Table 3 are then introduced in the mixer and the whole is then mingled for about 30 minutes.

Stearic acid can then be added depending on the composition.

The compositions are obtained according to the ingredients described in Table 3 below.

TABLE 3

| | A1 | A2 | A3 | A4 | A5 |
|---|---|---|---|---|---|
| Intermediate product | 200 | 200 | 200 | 200 | 200 |
| containing benzoyl peroxide (g) | | | | | |
| Sipernat ® 2200 (g) | 51.2 | 53.2 | 47.5 | — | 25.8 |
| Sipernat ® D17 | — | 18 | 23.7 | 80.2 | 25 |
| Stearic acid (g) | — | — | | — | 30 |

Each obtained composition comprises the following content of dibenzoyl peroxide relative to the total weight of the formulation:

TABLE 4

| | A1 | A2 | A3 | A4 | A5 |
|---|---|---|---|---|---|
| benzoyl peroxide wt % in | 54 | 50 | 50 | 48.4 | 48.3 |
| the final composition | | | | | |

b. Results

The compositions detailed in Table 3 are powdered compositions which are visually flowable.

### Flowability

The pourability (flowability) of such compositions is determined by carrying out a Pfrengle test, according to DIN 53 916.

150 ml of each sample are poured into a funnel whose outlet opening has been closed beforehand.

Then the opening is released so that the powder runs out, stirring slowly with the crank. After two minutes of rest, the height of the powder cone is measured using the measuring bar and read off the scale. The thickness of the base plate (25 mm) is to be subtracted from the measured height of the powder cone.

The result is the height of the powder cone in cm, minus 25 mm from the base disc.

The results are given in Table 5:

TABLE 5

| | A1 | A2 | A3 | A4 | A5 |
|---|---|---|---|---|---|
| Flowability, visually | Good | Very | Very | Good | Good |
| | | good | good | | |
| Flowability, | 2.5 | 2.7 | 2.6 | 2.9 | 2.9-2.7 |
| Pfrengle test (cm) | | | | | |

During the flowability test, one can contemplate that the powdered compositions have the ability of trickling out by themselves without mixing support.

### Dustiness

The compositions generate little to no dust.

One can additionally notice that compositions A5 is less dusty than compositions A1-A4 thanks to the presence of stearic acid.

1. A composition in powder form comprising:
at least one solid organic peroxide,
at least one inert filler,
at least one organic solvent, and
an aqueous phase.

2. The composition according to claim 1, wherein the solid organic peroxide is selected from the group consisting of solid diacyl peroxides, solid peroxydicarbonates, solid ketone peroxides, solid peroxy esters, solid hydroperoxides, solid dialkyl peroxides and mixtures thereof.

3. The composition according to claim 1, wherein the inert filler is soluble in an acrylic and/or polyester resin at a temperature ranging from 15° C. to 27° C.

4. The composition according to claim 1, wherein the inert filler is selected from the group consisting of silicas.

5. The composition according to claim 1, wherein the inert filler is selected from the group consisting of hydrophobic silicas, hydrophilic silicas, and mixtures thereof.

6. The composition according to claim 1, wherein the organic solvent is selected from the group consisting of alkyl diacid esters, cyclohexanedicarboxylates, esters based on glycols, polyglycols and polyhydric alcohols, and mixtures thereof.

7. The composition according to claim 1, comprising at least a crystalline powder having a melt temperature ranging from 30° C. to 80° C., especially a solid fatty acid.

8. The composition according to claim 1, wherein the total amount of aqueous phase present in said composition ranges from 3% to 10% by weight relative to the total weight of the composition.

9. A method for preparing a composition in powder form comprising the steps of:
a) mingling at least one organic solvent, at least one solid organic peroxide, and an aqueous phase,
b) reducing the aqueous phase from said mixture,
c) mixing at least one inert filler with the ensuing composition in order to obtain a powdered composition comprising said solid organic peroxide.

10. The method according to claim 9, further comprising a step d) of adding at least a crystalline powder having a melt temperature ranging from 30° C. to 80° C.

11. A composition in powder form obtained from the method as defined in claim 9.

12. A method comprising using the composition as defined in claim 1 as polymerization initiator for acrylic resins or unsaturated polyester resins, or as a polymer modifier.

13. A method comprising using the composition as defined in claim 1 for road marking, chemical anchoring, water proofing, and/or floor coating applications.

14. The composition of claim 1, the composition comprising at least two inert fillers.

15. The composition of claim 1, wherein the solid organic peroxide is selected from the group consisting of solid diacyl peroxides, solid peroxydicarbonates and mixtures thereof.

16. The composition of claim 1, wherein the solid organic peroxide is selected from the group consisting of solid diacyl peroxides.

17. The composition according to claim 1, wherein the inert filler is selected from a combination of at least one hydrophobic silica and at least one hydrophilic silica.

18. The composition according to claim 1, wherein the organic solvent is selected from the group consisting of cyclohexanedicarboxylates and alkyl diacid esters and mixtures thereof.

19. The composition according to claim 1, comprising a solid fatty acid.
